SEQUENCE LISTING (84 sequences)

SEQ ID NO: 1 - DNA, 1511 bp, Infectious bursal disease virus
  modified_base (1)..(131): a, c, g, t, unknown, or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaacctcaagttcgatcagatgctcctgactgcccagaacctaccggccagttacaactactgcaggctagtgagtcggagtctcacagtgaggtcaagcacactccctggtggcgtttatgcactaaacggcaccataaacgccgtgaccttccaaggaagcctgagtgaactgacagatgtaagctacaatgggttgatgtctgcaacagccaacatcaacgacaaaattgggaatgtcctagtaggggaaggggtcaccgtcctcagcttacccacatcatatgatcttgggtatgtgaggcttggtgaccccattcccgctatagggcttgacccaaaaatggtagccacatgtgacagcagtgacaggcccagagtctacaccataactgcagccgatgattaccaattctcatcacagtaccaaccaggtggggtaacaatcacactgttctcagccaacattgatgctatcacaagcctcagcgttgggggagagctcgtgtttcaaacaagcgtccaaggtcttgtactgggcgccaccatctactttataggctttgatgggactacggtaatcaccagggctgtggccgcagacaatgggctgacggccggcaccgacaatcttatgccattcaatcttgtgattccaaccaacgagataacccagccaatcacatccatcaaactggaggtagtgacctccaaaagtggtggccaggcaggggatcagatgtcatggtcggcaagtgggagcctagcagtgacgatccacggtggcaactatccaggggccctccgtcccgtcacactagtagcctacgaaagagtggcaacaggatccgtcgttacggtcgctggggtgagcaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacaccgactttcgtgagtacttcatggaggtggccgacctcaattctcccctgaagattgcaggagcatttggcttcaaagacataatccgggccataaggaggatagctgtgccggtggtctct

SEQ ID NO: 2 - DNA, 7 bp, Artificial Sequence
  Description of Artificial Sequence Synthetic probe
taatatc

SEQ ID NO: 3 - DNA, 7 bp, Artificial Sequence
  Description of Artificial Sequence Synthetic probe
gatatta

SEQ ID NO: 4 - DNA, 18 bp, Artificial Sequence
  Description of Artificial Sequence Synthetic probe
ctcagctaatatcgatgc

SEQ ID NO: 5 - DNA, 23 bp, Artificial Sequence
  Description of Artificial Sequence Synthetic probe
aggtggggtaacaatcacactgt

SEQ ID NO: 6 - DNA, 12 bp, Artificial Sequence
  Description of Artificial Sequence Synthetic probe
atactgggtgct

SEQ ID NO: 7 - DNA, 12 bp, Artificial Sequence
  Description of Artificial Sequence Synthetic probe
agcacccagtat

SEQ ID NO: 8 - DNA, 21 bp, Artificial Sequence
  Description of Artificial Sequence Synthetic probe
cttatactgggtgctaccatc

SEQ ID NO: 9 - DNA, 26 bp, Artificial Sequence
  Description of Artificial Sequence Synthetic probe
ccttataggctttgatgggactgcgg

SEQ ID NO: 10 - DNA, 18 bp, Artificial Sequence
  Description of Artificial Sequence Synthetic primer
gcccagagtctacaccat

SEQ ID NO: 11 - DNA, 18 bp, Artificial Sequence
  Description of Artificial Sequence Synthetic primer
cccggattatgtctttga

SEQ ID NO: 12 - DNA, 18 bp, Infectious bursal disease virus
ctcagctaatatcgatgc

SEQ ID NO: 13 - DNA, 18 bp, Infectious bursal disease virus
ctcagctaatatcgatgc

SEQ ID NO: 14 - DNA, 18 bp, Infectious bursal disease virus
ctcagctaatatcgatgc

SEQ ID NO: 15 - DNA, 18 bp, Infectious bursal disease virus
ctcagctaatatcgatgc

SEQ ID NO: 16 - DNA, 18 bp, Infectious bursal disease virus
ctcagctaatatcgatgc

SEQ ID NO: 17 - DNA, 18 bp, Infectious bursal disease virus
ctcagctaatatcgatgc

SEQ ID NO: 18 - DNA, 18 bp, Infectious bursal disease virus
ctcagctaatatcgatgc

SEQ ID NO: 19 - DNA, 18 bp, Infectious bursal disease virus
ctcagctaatatcgatgc

SEQ ID NO: 20 - DNA, 18 bp, Infectious bursal disease virus
ctcagctaatatcgatgc

SEQ ID NO: 21 - DNA, 18 bp, Infectious bursal disease virus
ctcagctaatatcgatgc

SEQ ID NO: 22 - DNA, 18 bp, Infectious bursal disease virus
ctcagctaatatcgatgc

SEQ ID NO: 23 - DNA, 18 bp, Infectious bursal disease virus
ctcagctaatattgatgc

SEQ ID NO: 24 - DNA, 18 bp, Infectious bursal disease virus
ctcagctaatatcgatgc

SEQ ID NO: 25 - DNA, 18 bp, Infectious bursal disease virus
ctcagctaatatcgatgc

SEQ ID NO: 26 - DNA, 18 bp, Infectious bursal disease virus
ctcagctaatatcgatgc

SEQ ID NO: 27 - DNA, 18 bp, Infectious bursal disease virus
ctcagctaatatcgatgc

SEQ ID NO: 28 - DNA, 18 bp, Infectious bursal disease virus
ctcagctaatatcgatgc

SEQ ID NO: 29 - DNA, 18 bp, Infectious bursal disease virus
ctcagccaacattgatgc

SEQ ID NO: 30 - DNA, 18 bp, Infectious bursal disease virus
ctcagccaacattgatgc

SEQ ID NO: 31 - DNA, 18 bp, Infectious bursal disease virus
ctcagccaacattgatgc

SEQ ID NO: 32 - DNA, 18 bp, Infectious bursal disease virus
ctcagccaacattgatgc

SEQ ID NO: 33 - DNA, 18 bp, Infectious bursal disease virus
ctcagccaacattgatgc

SEQ ID NO: 34 - DNA, 18 bp, Infectious bursal disease virus
ctcagccaacattgatgc

SEQ ID NO: 35 - DNA, 18 bp, Infectious bursal disease virus
ctcagccaacattgatgc

SEQ ID NO: 36 - DNA, 18 bp, Infectious bursal disease virus
ctcagccaacattgatgc

SEQ ID NO: 37 - DNA, 18 bp, Infectious bursal disease virus
ctcagccaacattgatgc

SEQ ID NO: 38 - DNA, 18 bp, Infectious bursal disease virus
ctcagccaacattgatgc

SEQ ID NO: 39 - DNA, 18 bp, Infectious bursal disease virus
ctcagccaacattgatgc

SEQ ID NO: 40 - DNA, 18 bp, Infectious bursal disease virus
ctcagccaacattgatgc

SEQ ID NO: 41 - DNA, 18 bp, Infectious bursal disease virus
ctcagccaacattgatgc

SEQ ID NO: 42 - DNA, 18 bp, Infectious bursal disease virus
ctcagccaacattgatgc

SEQ ID NO: 43 - DNA, 18 bp, Infectious bursal disease virus
ctcagccaacattgatgc

SEQ ID NO: 44 - DNA, 18 bp, Infectious bursal disease virus
ctcagccaacattgatgc

SEQ ID NO: 45 - DNA, 18 bp, Infectious bursal disease virus
ctcagccaacattgacgc

SEQ ID NO: 46 - DNA, 18 bp, Infectious bursal disease virus
ctcagccaacattgatgc

SEQ ID NO: 47 - DNA, 18 bp, Infectious bursal disease virus
ctcagccaacattgatgc

SEQ ID NO: 48 - DNA, 21 bp, Infectious bursal disease virus
cttatactgggtgctaccatc

SEQ ID NO: 49 - DNA, 21 bp, Infectious bursal disease virus
cttatactgggtgctaccatc

SEQ ID NO: 50 - DNA, 21 bp, Infectious bursal disease virus
cttatactgggtgctaccatc

SEQ ID NO: 51 - DNA, 21 bp, Infectious bursal disease virus
cttatactgggtgctaccatc

SEQ ID NO: 52 - DNA, 21 bp, Infectious bursal disease virus
cttatactgggtgctaccatc

SEQ ID NO: 53 - DNA, 21 bp, Infectious bursal disease virus
cttatactgggtgctaccatc

SEQ ID NO: 54 - DNA, 21 bp, Infectious bursal disease virus
cttatactgggtgctaccatc

SEQ ID NO: 55 - DNA, 21 bp, Infectious bursal disease virus
attatactgggtgctaccatc

SEQ ID NO: 56 - DNA, 21 bp, Infectious bursal disease virus
cttatactgggtgctaccatc

SEQ ID NO: 57 - DNA, 21 bp, Infectious bursal disease virus
cttatactgggtgctaccatc

SEQ ID NO: 58 - DNA, 21 bp, Infectious bursal disease virus
cttatactgggtgctaccatc

SEQ ID NO: 59 - DNA, 21 bp, Infectious bursal disease virus
cttatactgggcgctaccatc

SEQ ID NO: 60 - DNA, 21 bp, Infectious bursal disease virus
cttatcctgggtgctaccatc

SEQ ID NO: 61 - DNA, 21 bp, Infectious bursal disease virus
cttatcctgggtgctaccatc

SEQ ID NO: 62 - DNA, 21 bp, Infectious bursal disease virus
cttatactgggagctaccatc

SEQ ID NO: 63 - DNA, 21 bp, Infectious bursal disease virus
cttatactgggtgccaccatc

SEQ ID NO: 64 - DNA, 21 bp, Infectious bursal disease virus
cttatactgggtgctactatc

SEQ ID NO: 65 - DNA, 21 bp, Infectious bursal disease virus
cttgtactgggcgccaccatc

SEQ ID NO: 66 - DNA, 21 bp, Infectious bursal disease virus
cttgtactgggcgccaccatc

SEQ ID NO: 67 - DNA, 21 bp, Infectious bursal disease virus
cttgtactgggcgccaccatc

SEQ ID NO: 68 - DNA, 21 bp, Infectious bursal disease virus
cttgtactgggcgccaccatc

SEQ ID NO: 69 - DNA, 21 bp, Infectious bursal disease virus
cttgtactgggcgccaccatc

SEQ ID NO: 70 - DNA, 21 bp, Infectious bursal disease virus
cttgtactgggcgccaccatc

SEQ ID NO: 71 - DNA, 21 bp, Infectious bursal disease virus
cttgtactgggcgccaccatc

SEQ ID NO: 72 - DNA, 21 bp, Infectious bursal disease virus
cttgtactgggcgccaccatc

SEQ ID NO: 73 - DNA, 21 bp, Infectious bursal disease virus
cttgtactgggcgccaccatc

SEQ ID NO: 74 - DNA, 21 bp, Infectious bursal disease virus
cttgtactgggtgccaccatc

SEQ ID NO: 75 - DNA, 21 bp, Infectious bursal disease virus
cttgtactgggcgccaccatc

SEQ ID NO: 76 - DNA, 21 bp, Infectious bursal disease virus
cttgtactgggcgccaccatc

SEQ ID NO: 77 - DNA, 21 bp, Infectious bursal disease virus
cttgtactgggcgccaccatc

SEQ ID NO: 78 - DNA, 21 bp, Infectious bursal disease virus
cttgtactgggcgccaccatc

SEQ ID NO: 79 - DNA, 21 bp, Infectious bursal disease virus
cttgtactgggcgccaccatc

SEQ ID NO: 80 - DNA, 21 bp, Infectious bursal disease virus
cttgtactgggcgccaccatc

SEQ ID NO: 81 - DNA, 21 bp, Infectious bursal disease virus
cttgtactgggcgccaccatc

SEQ ID NO: 82 - DNA, 21 bp, Infectious bursal disease virus
cttgtgctgggcgccaccatc

SEQ ID NO: 83 - DNA, 21 bp, Infectious bursal disease virus
cttgtactaggcgccaccatc

SEQ ID NO: 84 - RNA, 1511 nt, Infectious bursal disease virus
  modified_base (1)..(131): a, g, c, t, unknown, or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaugacaaaccugcaagaucaaacccaacagauuguuccguucauacggagccuucugaugccaacaaccggaccggcguccauuccggacgacacccuggagaagcacacucucaggucagagaccucgaccuacaauuugacugugggggacacagggucagggcuaauugucuuuuucccuggauucccuggcucaauugugggugcucacuacacacugcagagcaaugggaaccucaaguucgaucagaugcuccugacugcccagaaccuaccggccaguuacaacuacugcaggcuagugagucggagucucacagugaggucaagcacacucccugguggcguuuaugcacuaaacggcaccauaaacgccgugaccuuccaaggaagccugagugaacugacagauguaagcuacaauggguugaugucugcaacagccaacaucaacgacaaaauugggaauguccuaguaggggaaggggucaccguccucagcuuacccacaucauaugaucuuggguaugugaggcuuggugaccccauucccgcuauagggcuugacccaaaaaugguagccacaugugacagcagugacaggcccagagucuacaccauaacugcagccgaugauuaccaauucucaucacaguaccaaccaggugggguaacaaucacacuguucucagccaacauugaugcuaucacaagccucagcguugggggagagcucguguuucaaacaagcguccaaggucuuguacugggcgccaccaucuacuuuauaggcuuugaugggacuacgguaaucaccagggcuguggccgcagacaaugggcugacggccggcaccgacaaucuuaugccauucaaucuugugauuccaaccaacgagauaacccagccaaucacauccaucaaacuggagguagugaccuccaaaagugguggccaggcaggggaucagaugucauggucggcaagugggagccuagcagugacgauccacgguggcaacuauccaggggcccuccgucccgucacacuaguagccuacgaaagaguggcaacaggauccgucguuacggucgcuggggugagcaacuucgagcugaucccaaauccugaacuagcaaagaaccugguuacagaauacggccgauuugacccaggagccaugaacuacacaaaauugauacugagugagagggaccgucuuggcaucaagaccgucuggccaacaagggaguacaccgacuuucgugaguacuucauggagguggccgaccucaauucuccccugaagauugcaggagcauuuggcuucaaagacauaauccgggccauaaggaggauagcugugccgguggucucu

(19) United States
(12) Patent Application Publication
(10) Pub. No.: US 2006/0188871 A1
(43) Pub. Date: Aug. 24, 2006

(54) Detection of very virulent infectious bursal disease virus

(76) Inventor: Daral J. Jackwood, Wooster, OH (US)

Correspondence Address: CALFEE HALTER & GRISWOLD, LLP, 800 SUPERIOR AVENUE, SUITE 1400, CLEVELAND, OH, 44114, US

(21) Appl. No.: 11/066,130

(22) Filed: Feb. 24, 2005

(51) Int. Cl. C12Q 1/70; C12Q 1/68

(52) U.S. Cl. 435/5; 435/6

(57) ABSTRACT

Methods of identifying animals infected with a vvIBDV are provided. The methods comprise contacting a nucleic acid sample obtained from the animal or a nucleic acid product obtained by amplifying RNA obtained from the animal with one or more probe pairs, each of which comprises a mutation probe and an anchor probe, and then determining the melting temperature of any hybridization complex that is formed when the one or more probe pairs hybridize with a nucleic acid in the sample. Such determination is made using FRET analysis. In one embodiment the mutation probe comprises a sequence identical to a first mutated target sequence of SEQ ID NO: 1 in which the cytosine at position 827 is substituted with a thymidine, the cytosine at position 830 is substituted with a thymidine, and the thymidine at position 833 is substituted with a cytosine, or the reverse complement thereof. In another embodiment, the mutation probe comprises a sequence identical to a second mutated target sequence of SEQ ID NO: 1 in which the guanine at position 897 is substituted with an adenine, the cytosine at position 905 is substituted with a thymidine, and the cytosine at position 908 is substituted with an thymidine. Also, provided herein are kits containing nucleotide probe pairs that can be used in the present methods.

### FIELD OF THE INVENTION

The present invention relates to novel methods of detecting very virulent infectious bursal disease virus in nucleic acid samples.

### BACKGROUND

Infectious bursal disease (IBD) is an immunosuppressive disease that occurs in young chickens. The etiologic agent, infectious bursal disease virus (IBDV), exists naturally in several antigenic and pathogenic forms. The pathogenic forms of the virus range from attenuated to very virulent (vvIBDV). All appear to cause some degree of damage to the immune system. The vvIBDV strains were first described in the late 1980's and were identified as causing an acute form of the disease characterized by high morbidity and mortality in susceptible chicken flocks (Van Den Berg, T. P. Acute infectious bursal disease in poultry: a review. Avian Pathology 29: 175-194. 2000.).

The very virulent phenotype of IBDV was first discovered in Europe (Domanska, K., et al. Antigenic and genetic diversity of early European isolates of Infectious bursal disease virus prior to the emergence of the very virulent viruses: early European epidemiology of Infectious bursal disease revisited? Archives of Virology 149: 465-480. 2004, Van Den Berg, T. P. Acute infectious bursal disease in poultry: a review. Avian Pathology 29: 175-194. 2000). It quickly spread to Asia and Japan where it was described in the early 1990's (Van Den Berg, T. P. Acute infectious bursal disease in poultry: a review. Avian Pathology 29: 175-194. 2000). In 1995, during the 63^{rd} General Session of the Office of International des Epizooties (OIE), 80% of members countries reported acute cases of IBD (Van Den Berg, T. P. Acute infectious bursal disease in poultry: a review. Avian Pathology 29: 175-194. 2000). Although vvIBDV has been identified on nearly every continent of the world, it has yet to be found in North America, Australia and New Zealand. There is a real and immediate concern that the very virulent form of IBDV will continue to spread until it is present on every continent.

Early detection is critical to controlling acute IBD (Van Den Berg, T. P. Acute infectious bursal disease in poultry: a review. Avian Pathology 29: 175-194. 2000). Surveillance programs are not being used because a rapid and economical assay for the reliable detection of markers for vvIBDV strains has not been developed. RT/PCR-RFLP assays to identify a restriction enzyme marker (SspI) for the vvIBDV phenotype have been described (Ikuta, N., et al. Molecular Characterization of Brazillian Infectious Bursal Disease Viruses. Unknown. 2000, Jackwood, D. J. and S. E. Sommer. Restriction Fragment Length Polymorphisms in the VP2 Gene of Infectious Bursal Disease Viruses from Outside the United States. Avian Diseases 43: 310-314. 1999, Lin, Z., et al. Sequence comparisons of a highly virulent infectious bursal disease virus prevalent in Japan. Avian Diseases 37: 315-323. 1993). However, this assay is expensive and not practical for testing large numbers of samples. In addition, the SspI marker has been found in some IBDV strains that do not exhibit the very virulent phenotype (Banda, A., et al. Molecular Characterization of Seven Field Isolates of Infectious Bursal Disease Virus Obtained from Commercial Broiler Chickens. Avian Diseases 45: 620-630. 2001), so its specificity is questionable. Accordingly, additional methods for detecting the presence of vvIBDV in animals is desirable. Methods that are rapid and reliable, and that can be used to test large numbers of samples are particularly. desirable.

### SUMMARY OF THE INVENTION

The present invention provides methods of identifying animals infected with a vvIBDV. The method comprises contacting a nucleic acid sample obtained from the animal or a nucleic acid product obtained by amplifying RNA obtained from the animal with one or more oligonucleotide probe pairs, each of which comprises a mutation probe and an anchor probe, and then determining the temperature at which the one or more mutation probes disassociate from a hybridization complex that is formed when the one or more probe pairs hybridize with a nucleic acid in the sample. Results in which the melting temperature (Tm) of the hybridization complex formed between the mutation probe and a nucleic acid in the sample is greater than the melting temperature of a hybridization complex formed when the mutation probe is hybridized with a nucleic acid comprising SEQ ID NO: 1, or the reverse complement thereof, and/or is within 4° C. of the melting temperature of the melting temperature of a hybridization complex that is formed when the mutation probe and anchor probe are hybridized with a nucleic acid sample comprising their target sequences indicates that the animal is or has been infected with vvIBDV.

In one embodiment the mutation probe comprises a sequence identical to a first mutated target sequence of SEQ ID NO:1 in which the cytosine at position 827 is substituted with a thymidine, the cytosine at position 830 is substituted with a thymidine, and the thymidine at position 833 is substituted with a cytosine, or the reverse complement thereof. In this embodiment, the anchor probe targets a sequence upstream of the mutated target sequence. In another embodiment, the mutation probe comprises a sequence identical to a second mutated target sequence of SEQ ID NO: 1 in which the guanine at position 897 is substituted with an adenine, the cytosine at position 905 is substituted with a thymidine, and the cytosine at position 908 is substituted with an thymidine. In this embodiment, the anchor probe targets a sequence downstream of the second mutated target sequence. The temperature at which each mutation probe disassociates from the hybridization complex is determined by fluorescence resonance energy transfer (FRET) analysis.

The present invention also relates to kits comprising one or more of the oligonucleotide probe pairs that can be used in the present methods, and to methods of using such kits to determine if a nucleic acid sample comprises all or a portion of the VP2 gene of a vvIBDV.

It is to be understood that both the foregoing general description and the following detailed description are exemplary and explanatory only and are not restrictive of the invention, as claimed.

The accompanying drawings, which are incorporated in and constitute a part of this specification, may illustrate embodiments of the invention, and together with the description, serve to explain the principles of the invention.

### BRIEF DESCRIPTION OF THE DRAWINGS

FIG. 1 shows the nucleotide sequence, SEQ ID NO: 1, of the sense strand of the VP2 gene of a non-very virulent STC strain of IBDV. The sequence, Gen Bank accession number D00499 was first reported in Kibenge et al., J. Gen. Virol. 71:569-571, 1990.

FIG. 2 shows the nucleotide sequence, SEQ ID NO: 4, of the vv232 probe as compared to the same region of vvIBDV and non-vvIBDV strains. Nucleotides that differ from the probe sequence are in bold type.

FIG. 3 shows the nucleotide sequence, SEQ ID NO: 8 of the vv256 probe as compared to the same region of vvIBDV and non-vvIBDV strains. Nucleotides that differ from the probe sequence are in bold type.

FIG. 4 shows the target site of two embodiments of the first mutation probe, wherein one embodiment comprises the sequence TAATATC, SEQ ID NO: 2 and the other embodiment comprises the sequence GATATTA, SEQ ID. NO: 3, in relation to the target site of the first anchor probe; and the target site of two embodiments of the second mutation probe, wherein one embodiment comprises the sequence ATACTGGGTGCT, SEQ. ID NO: 6, and the other embodiment comprises the sequence AGCACCCAGTAT, SEQ ID NO: 7, in relation to the target site of the second anchor probe.

### DESCRIPTION OF THE EMBODIMENTS

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The present invention is based, at least in part, on the discovery that nucleic acid samples containing the double-stranded RNA genome of a vvIBDV or the VP2 gene of a vvIBDV can be easily and rapidly distinguished from nucleic acid samples containing the double-stranded RNA genome of non-very virulent strains of IBDV using FRET analysis, melting temperature analysis, and mutation probes and anchor probes directed at specific regions of the VP2 gene of vvIBDV.

As used herein, “nucleic acid” may refer to either DNA or RNA, or molecules which contain both deoxy- and ribonucleotides. By “nucleic acid” or “oligonucleotide” or grammatical equivalents herein means at least two nucleotides covalently linked together. As used herein, “nucleic acid” encompasses both double stranded and single-stranded nucleic acid molecules. A nucleic acid or oligonucleotide of the present invention will generally contain phosphodiester bonds, although in some cases, nucleic acid analogs, having modifications well known in the art, are also included. Modifications of the ribose-phosphate backbone may be done to facilitate the addition of additional moieties such as labels, or to increase the stability and half-life of such molecules in various environments. In one embodiment the oligonucleotide comprises peptide nucleic acids (PNA), the backbones of which are substantially non-ionic under neutral conditions, in contrast to the highly charged phosphodiester backbone of naturally occurring nucleic acids.

Methods of Identifying Animals Infected with vvIBDV

Provided herein are methods for determining whether an animal, particularly an avian species, is infected with vvIBDV. In one embodiment, the animal is a chicken. The method comprises contacting a nucleic acid sample obtained from the animal or a nucleic acid product obtained by amplifying RNA obtained from the animal with at least one probe pair comprising an oligonucleotide probe, referred to hereinafter as the “mutation probe”, that is complementary to a target sequence in a specific mutation locus in the VP2 gene of vvIBDV and at least one oligonucleotide probe, referred to hereinafter as the “anchor probe”, that is complementary to a target sequence in an anchor locus adjacent to or within a few base pairs of the mutation locus. The temperature at which the anchor probes of the present invention disassociate from their target sequences is at least 4° C. greater than the temperature at which the mutation probes of the present invention disassociate from their target sequences. One member of the oligonucleotide probe pair is labeled with a fluorescence energy transfer donor, and the other member of the probe pair is labeled with an fluorescence energy transfer acceptor. The probe pair is contacted with the nucleic acid sample under conditions that permit each member of the probe pair to hybridize with at least one strand of a nucleic acid in the test sample to provide a hybridization complex between the probe pair and the nucleic acid. Then, the melting temperature of the hybridization complex, i.e., the temperature at which the mutation probe disassociates from the nucleic acid is determined by fluorescence resonance energy transfer (FRET) analysis. Results in which the melting temperature (Tm) of the hybridization complex formed between the mutation probe and a nucleic acid in the sample is greater than the melting temperature of a hybridization complex (referred to hereinafter as the “non-vvIBDV control hybridization complex)” formed when the mutation probe is hybridized with a nucleic acid comprising SEQ ID NO: 1, or the reverse complement thereof, indicates that the sample comprises the VP2 gene, or a portion thereof, of a vvIBDV. In certain embodiments, the melting temperature of the hybridization complex that is formed between the mutation probe and a nucleic acid in the test sample is compared to the melting temperature of a hybridization complex (referred to hereinafter as the “vvIBDV control hybridization complex”) that is formed when the mutation probe and anchor probe are hybridized with a nucleic acid comprising their target sequences. Results in which the melting temperature of the hybridization complex formed between the inventive probes and the test sample are within 4° C. of the melting temperature of the vvIBDV control hybridization complex indicates that the sample comprises at least one strand of the VP2 gene, or a portion thereof, of a vvIBDV.

Oligonucleotide Probe Pairs

In certain embodiments the present methods employ a first mutation probe designed to hybridize to target sequence in a first mutation locus in the VP2 gene of vvIBDV and a first anchor probe designed to hybridize to a target sequence in a first anchor locus adjacent to or within a few nucleotides upstream of the mutation probe target sequence. In certain embodiments, the first mutation probe comprises a sequence identical to a first mutated target sequence of SEQ ID NO: 1 in which the cytosine at position 827 is substituted with a thymidine, the cytosine at position 830 is substituted with a thymidine, and the thymidine at position 833 is substituted with a cytosine. In another embodiment, the first mutation probe of the present invention is the reverse complement of the first mutated target sequence.

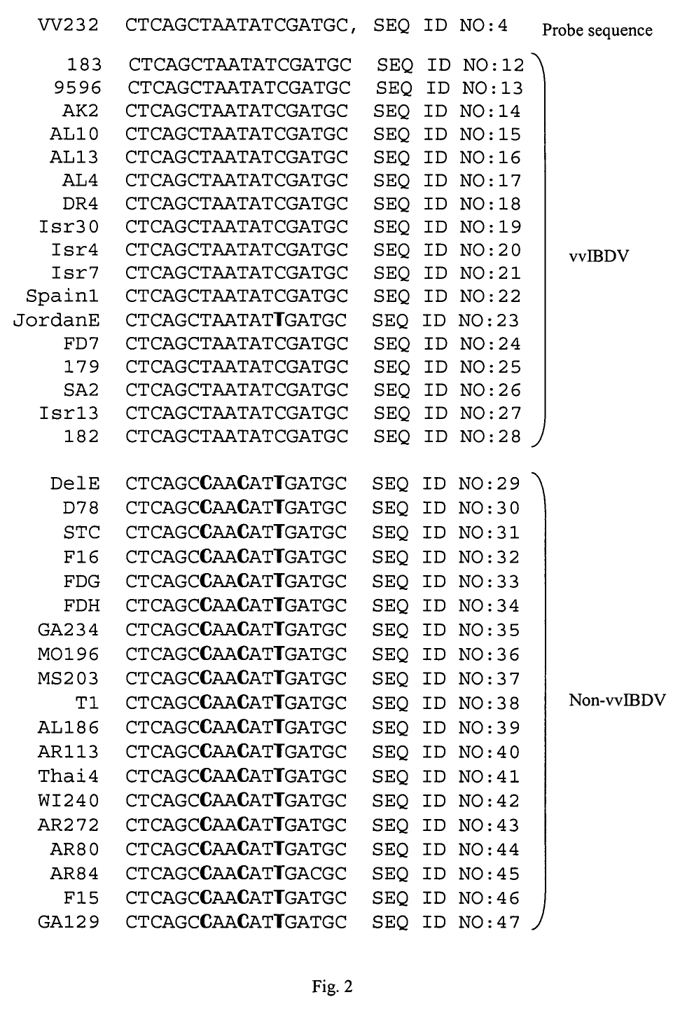

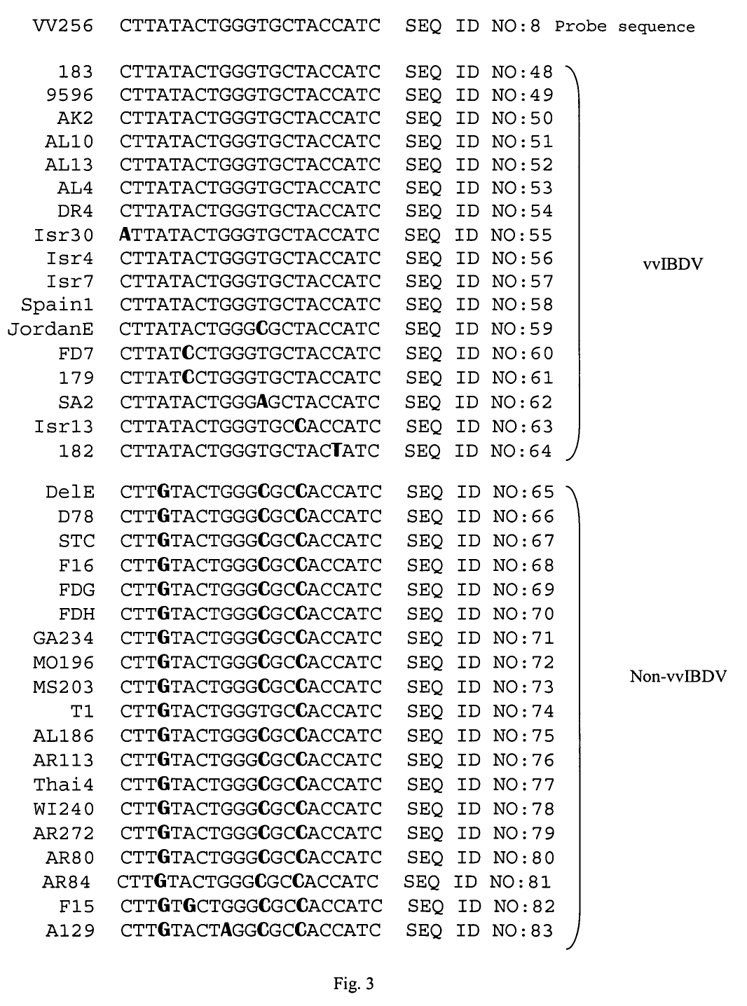

In certain embodiments, the first mutation probe comprises the sequence TAATATC, SEQ ID NO: 2. In other embodiments, the first mutation probe comprises the sequence GATATTA, SEQ ID NO: 3. In certain embodiments, the first mutation probe is from 12 to 25 nucleotides in length and comprises all or a portion of the vv232 mutation probe sequence, SEQ ID NO: 4, shown in FIG. 2, provided that the portion comprises SEQ ID NO: 2, or all or a portion of the reverse complement of SEQ ID NO: 4, provided that the portion of the reverse complement comprises SEQ ID NO: 3. In certain embodiments, the first mutation probe comprises from 12 to 18 contiguous nucleotides of SEQ ID NO: 4, or the reverse complement thereof. In other embodiments, the first mutation probe comprises from 12 to 17 contiguous nucleotides of SEQ ID. NO: 4, and from 1 to 13 contiguous nucleotides that lie upstream of nucleotide 827 and/or downstream of nucleotide 833 of SEQ ID NO: 1, or the reverse complement thereof.

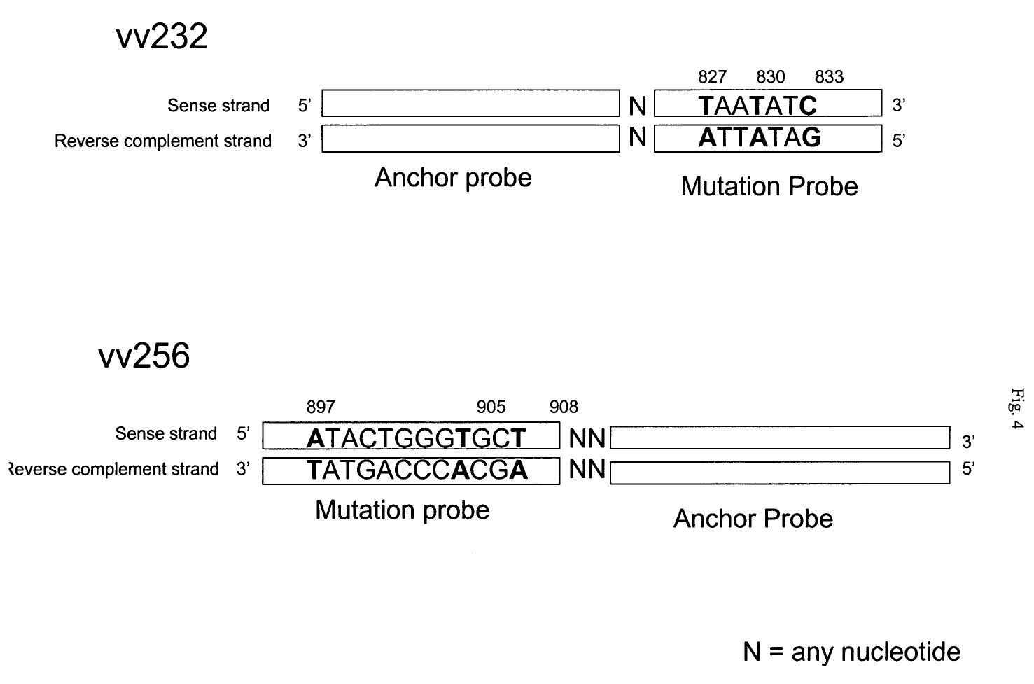

Methods that employ the first mutation probe also employ an anchor probe, referred to hereinafter as the “first anchor probe”, designed to hybridize to a sequence in an anchor locus that is adjacent to or within a few base pairs upstream of the first mutation locus. (See FIG. 4.) The anchor probe is 12 or more nucleotides in length and disassociates from its target sequence at a temperature at least 4° C. higher than the temperature at which the first mutation probe disassociates from its target sequence. In one embodiment, the first anchor probe has a sequence that is identical to a sequence that is upstream of nucleotide 827 in the first mutated target sequence. In other embodiments, the first anchor probe has a sequence that is the reverse complement of a sequence that is upstream of nucleotide 827 of the first mutated target sequence. In certain embodiments, the first anchor probe is 12 or more nucleotides in length and comprises from 12-23 contiguous nucleotides of the vv232 anchor probe sequence, SEQ ID NO: 5, shown in Table 2, or the reverse complement thereof.

In certain embodiments, the present methods employ a second mutation probe designed to hybridize to a second mutated target sequence in a second mutation locus in the VP2 gene of vvIBDV and a second anchor probe designed to hybridize to a target sequence in a second anchor locus adjacent to or within a few nucleotides downstream of the second mutation locus. (See FIG. 4.) In certain embodiments, the second mutation probe comprises a sequence identical to a second mutated target sequence of SEQ ID NO: 1 in which the guanine at position 897 is substituted with an adenine, the cytosine at position 905 is substituted with a thymidine, and the cytosine at position 908 is substituted with an thymidine. In another embodiment, the second mutation probe of the present invention is the reverse complement of the second mutated sequence. In certain embodiments, the second mutation probe comprises the sequence ATACTGGGTGCT, SEQ ID NO: 6. In other embodiments the second mutation probe comprises the sequence AGCACCCAGTAT, SEQ ID NO: 7. In certain embodiments, the second mutation probe is from 12 to 25 nucleotides in length and comprises all or a portion of the vv256 mutation probe sequence, SEQ ID NO: 8, shown in FIG. 2, provided that the portion comprises SEQ ID NO: 6, or all or a portion of the reverse complement of SEQ ID NO: 8, provided that the portion comprises SEQ ID NO: 7. In certain embodiments, the second mutation probe comprises from 12 to 20 contiguous nucleotides of SEQ ID NO: 8, or the reverse complement thereof. In other embodiments, the second mutation probe comprises from 12 to 19 contiguous nucleotides of SEQ ID. NO: 8, and from 1 to 13 of the nucleotides that lie upstream of nucleotide 897 and/or downstream of nucleotide 908 of SEQ ID NO: 1, or the reverse complement thereof.

Methods that employ the second mutation probe also employ an anchor probe, referred to hereinafter as the “second anchor probe”, designed to hybridize to a target sequence in a second anchor locus that is downstream and adjacent to or within a few nucleotides of the second mutation locus in the VP2 gene of vvIBDV. (See FIG. 4) In certain embodiments, the second anchor probe is 12 or more nucleotides in length and comprises from 12-23 contiguous nucleotides of the vv256 anchor probe sequence, SEQ ID NO: 9, shown in Table 2.

In certain embodiments, the nucleic acid test sample is contacted with the first oligonucleotide probe pair and the second oligonucleotide probe pair and the temperatures at which the first mutation probe and the second mutation probe disassociate from the first hybridization complex and the second hybridization complex, respectively, are determined.

The anchor probes of the present invention are designed to disassociate from a hybridization complex comprising the anchor probe and its target sequence at a temperature at least 4° C. higher than the temperature at which the mutation probe disassociates from a hybridization complex comprising the mutation probe and its target sequence. Thus, the melting temperature of a hybridization complex comprising the anchor probe and its target sequence can be 4, 5, 6, 7, 8, 9, 10 or even more degrees higher than the melting temperature of a hybridization complex comprising the mutation probe and its target sequence. Probe melting temperature is dependent upon external factors (salt concentration and pH) and intrinsic factors (concentration, duplex length, GC content and nearest neighbor interactions) (Wetmur, Crit. Rev. Biochem. Mol. Biol. 26:227-259 (1991); Wetmur, In: Meyers, R A, ed. Molecular Biology and Biotechnology, VCH, New York, pp. 605-608 (1995); Brown et al. J Mol. Biol. 212:437-440 (1990); Gaffney et al., Biochemistry 28:5881-5889 (1989)).

The methods of the invention involve combining fluorescently labeled oligonucleotide probes with the nucleic acid test sample such that oligonucleotide probes hybridize, which hybridization allows fluorescence resonance energy transfer between a donor fluorophore on one member of the probe pair and an acceptor fluorophore on the other member of the probe pair. The emission from the acceptor fluorophore is then measured at different increasing temperatures. The Tm is determined to be that temperature at which there is an abrupt reduction in emission. The color of the emission and the Tm are used to determine whether the test sample does or does not contain a nucleic acid comprising the first mutation locus and/or the second mutation locus.

Fluorescence resonance energy transfer (FRET) occurs between two fluorophores when they are in physical proximity to one another and the emission spectrum of one fluorophore overlaps the excitation spectrum of the other. The rate of resonance energy transfer is:
(8 785E⁻⁵)(t−1)(k²)(n⁻⁴)(q_{D})(R⁻⁶)(J._{DA}), where:
t=excited state lifetime of the donor in the absence of the acceptor;
k²=an orientation factor between the donor and acceptor;
n=refractive index of the visible light in the intervening medium;
q_{D}=quantum efficiency of the donor in the absence of the acceptor;
R=distance between the donor and acceptor measured in Angstroms;
J_{DA}=the integral of (F_{D})(e_{A})(W⁴) with respect to W at all overlapping wavelengths with:
F_{D}=peak normalized fluorescence spectrum of the donor;
A=molar absorption coefficient of the acceptor (M⁻¹ cm⁻¹);
W⁴=wavelength (nm).

For any given donor and acceptor, a distance where 50% resonance energy transfer occurs can be calculated and is abbreviated R₀. Because the rate of resonance energy transfer depends on the 6th power of the distance between donor and acceptor, resonance energy transfer changes rapidly as R varies from R₀. At 2 R₀, very little resonance energy transfer occurs, and at 0.5 R₀, the efficiency of transfer is nearly complete, unless other forms of de-excitation predominate.

Using the method of Wittwer et al. (1997), fluorescently labeled oligonucleotides have been designed to hybridize to the same strand of a DNA sequence, resulting in the donor and acceptor fluorophores being separated by a distance ranging from about 0 to about 25 nucleotides. In certain embodiments, the donor and acceptor fluorophores are separated by a distance ranging from about 0-5 nucleotides. In other embodiments, the donor and acceptor fluorophores are separated by a distance ranging from about 0-2 nucleotides. In another embodiment, the donor and acceptor fluorophores are separated by 1 nucleotide. When both of the fluorescently labeled oligonucleotides are not hybridized to their complementary sequence on the targeted DNA, then the distance between the donor fluorophore and the acceptor fluorophore is too great for resonance energy transfer to occur. Under these conditions, the acceptor fluorophore and the donor fluorophore do not produce a detectable increased fluorescence by the acceptor fluorophore.

Acceptable fluorophore pairs for use as fluorescent resonance energy transfer pairs are well known to those skilled in the art and include, but are not limited to, phycoerythrin as the donor and Cy7 as the acceptor, fluorescein as the donor in combination with any one of Cy5, Cy5.5, IRD 700, LC Red 640 and LC Red 705 as the acceptor. It is understood that any functional FRET donor/acceptor combination may be used in the invention. In certain embodiments, e.g. when the first set of probes and the second set of probes are added to separate PCR vials, the emission from each of the acceptor fluorophores may be the same. In other embodiments, e.g. when both sets of probes are added to the same PCR vial, the emission from each of the acceptor fluorophores preferably is different. Labeled probes can be constructed following the disclosures of, for example, Wittwer et al., BioTechniques 22:130-138, 1997; Lay and Wittwer, Clin. Chem. 43:2262-2267, 1997; and Bernard Pset al., Anal. Biochem. 255:101-107, 1998. Each of these disclosures is incorporated herein in its entirely. Suitable FRET acceptors include, but are not limited to, LC Red 640, Cy 5, Cy 5.5 and LC Red 705.

Preparation of the Sample

The nucleic acid sample used in the present methods, i.e., the nucleic acid test sample, can be a single-stranded or double-stranded nucleic acid. In certain embodiments, the nucleic acid test sample is a double-stranded RNA that has been isolated from a tissue, e.g. blood, muscle, etc. of an animal. In other embodiments, the nucleic acid sample is one of the strands of the isolated double-stranded RNA sample. A particularly useful sample is a dsRNA isolated from the bursa of a chicken. Methods for isolating RNA from tissue samples are known in the art. A method for isolating dsRNA from the bursa of a chicken is described in the Examples below. In another embodiment, the sample is a cDNA product that is formed by reverse transcriptase-polymerase chain reaction (RT-PCR) amplification of a single stranded or double-stranded RNA sample isolated from an animal. The cDNA molecule is prepared using RT-PCR techniques known in the art and primers that flank one or both of the present mutation and anchor loci within the VP2 gene of IBDV. One example of a useful primer pair is shown described in the Examples below.

Hybridization of the Probe Pairs to the Test Sample

The nucleic acid sample and the flourophore-labeled mutation probes and anchor probes are contacted under conditions that allow the mutation probes and anchor probes to hybridize with their target sequences and to form a hybridization complex. Suitable conditions include, but are not limited to, those provided in the LightCycler-RNA amplification kit for hybridization probes (Roche, Molecular Biochemicals, Alamedia, Calif.) where each reaction would contain 4 μl 5×RT-PCR reaction mix, 4.5 mM MgCl₂, 0.25 μM of each IBDV primer, 0.2 μM of each probe, 0.5 μl template nucleic acid and sterile H₂O added to a final reaction volume of 20 μl. Hybridization would occur at an annealing temperature of 61° C. or lower for 10 sec.

Determination of the Melting Temperature of the Hybridization Complexes

Formation of a hybridization complex comprising the mutation probe and a molecule in the nucleic acid sample is analyzed by FRET analysis, i.e., by detecting or measuring the fluorescence emitted by the test sample. Devices for measuring fluorescence emission are known in the art. A device for measuring FRET acceptor emission at two different wavelengths at varying temperatures is also commercially available (i.e., LightCycler™). Devices for simultaneously detecting FRET acceptor emission at more than two wavelengths at varying temperatures are described below.

In a certain embodiments of the invention, the emission of each FRET acceptor is measured at a different wavelength spectrum, preferably around its maximum emission wavelength, at a first temperature. This measurement is then repeated at a second temperature. In certain embodiments, such measurements are made repeatedly, preferably over a range of progressively increasing temperatures. The first measurement is made at a temperature low enough to ensure that each of the probes is hybridized. Generally, this temperature will be at least 20° C.

The melting temperature (Tm) of the resulting hybridization complexes is determined by measuring emissions at subsequently higher temperatures. Eventually, as the temperature is increased, the mutation probe will dissociate (melt) from the nucleic acid to which it is hybridized. This dissociation results in disruption of the FRET donor/acceptor association, which is seen as an abrupt drop in FRET acceptor emission.

In certain embodiments, FRET acceptor emission measurements are made every 50 to 10,000 msec. For example, FRET acceptor emission measurements can be made every 100 to 1,000 msec. In other embodiments, FRET acceptor emission measurements are made every 100-200 msec. The temperature can be varied by 0.01° C. per second to 5° C. per second. The temperature can be varied by 0.5° C. per second to 1° C. In certain embodiments, the temperature is varied by at least 0.5° C. per second.

### EXAMPLES

### Materials and Methods

Viruses. The vvIBDV strains used to develop and validate the present methods were submitted as genomic RNA to our laboratory under import permit #44226 from the USDA, Animal and Plant Health Inspection Service. The viruses were from Europe, Asia, Africa, the Caribbean and the Middle East. Genetic material from non-vvIBDV strains was obtained from domestic vaccines and outbreaks of infectious bursal disease (IBD) in the United States. These non-vvIBDV strains included variant and classic viruses. All viruses used in this study and their country of origin are listed in table 1.

TABLE 1
Virus samples and their geographic origin.

| Country of Origin | Virus Samples |
|---|---|
| USA^{A} | Del-E, D78, STC, F16, FDG, |
| | FDH, GA234, MO196, |
| | MS 203, T1, AL186, AR113, |
| | WI240, AR272, AR80, |
| | AR84, F15, GA129 |
| Israel^{B} | Isr1, Isr2, Isr3, Isr4, Isr5, Isr6, |
| | Isr7, Isr8, Isr9, Isr10, Isr11, |
| | Isr12, Isr13, Isr14, Isr15, Isr16, |
| | Isr17, Isr19, Isr20, Isr21, |
| | Isr23, Isr24, Isr25, Isr29, Isr30 |
| Singapore^{B} | 179, 182, 183 |
| Korea^{B} | 9596, 91108 |
| France^{B} | AK2, AL1, AL4, AL6, AL10, AL13, FD7 |
| Dominican | DR4 |
| Republic^{B} | |
| South Africa^{B} | SA2 |
| Spain^{B} | Spain1 |
| Jordan^{B} | Jordan E |
| Thailand^{B} | Thai4 |

^{A}All samples from the United States were non-vvIBDV strains and consisted of serotype 1 variant, classic and field isolates.
^{B}Samples from these countries were submitted as suspect vvIBDV strains.

Viral RNA extraction. Genomic RNA from IBDV samples originating outside the U.S. arrived at our laboratory after being treated with phenol and chloroform according to import permit #44226. These samples were rinsed twice with TNE buffer [10 mM Tris-HCl (pH 8.0), 100 mM NaCl, 1 mM ethylenediaminetetraacetic acid] before being treated with proteinase K (Sigma Chemical Co., St. Louis, Mo.) and acid phenol (pH 4.3) (AMRESCO, Solon, Ohio) using our standard procedures (Jackwood, D. J. and S. E. Sommer. Avian Diseases 41: 627-637. 1997). Genomic RNA from domestic IBDV strains was harvested from homogenized bursa tissue using proteinase K and acid phenol (Jackwood, D. J. and S. E. Sommer. Avian Diseases 41: 627-637. 1997).

Real-time RT-PCR. A LightCycler instrument (Roche Diagnostics, Indianapolis, Ind.) and LightCycler-RNA amplification kit for hybridization probes (Roche, Molecular Biochemicals, Alamedia, Calif.) were used. Each reaction contained 4 μl 5×RT-PCR reaction mix, 4.5 mM MgCl₂, 0.25 μM of each IBDV primer, 0.2 μM of each probe, 0.5 μl viral RNA and sterile H₂O was added to a final reaction volume of 20 μl. The primers amplifed a 743-bp region of VP2 (743-1: 5′-GCCCAGAGTCTACACCAT-3′, SEQ ID NO:10 and 743-2: 5′-CCCGGATTATGTCTTTGA-3′, SEQ ID NO: 11) (Jackwood, D. J. and S. E. Sommer. Avian Diseases 42: 321-339. 1998). The LightCycler reactions began with a reverse transcriptase incubation at 55° C. for 7 min, followed by a denaturation step at 95° C. for 5 min and 40 cycles of denaturation at 95° C. for 1 sec, annealing at 61° C. for 10 sec and elongation at 72° C. for 30 sec.

Probe design. The vvIBDV specific probes were designed using published sequences of vvIBDV strains isolated from different continents (Banda, A. and P. Villegas. Avian Diseases 48: 540-549. 2004, Brown, M. D. and M. A. Skinner. Virus Research 40: 1-15. 1996, Chen, H. Y., et al. Avian Diseases 42: 762-769. 1998, Domanska, K., et al. Archives of Virology 149: 465-480. 2004, Indervesh, A. K. et al. Acta virologica 47: 173-177. 2003, Kwon, H. et al. Avian Diseases 44: 691-696. 2000, Lin, Z., et al. Avian Diseases 37: 315-323. 1993, Liu, H. J., et al. Research in Veterinary Science 70: 139-147. 2001, Owoade, A. A., et al. Archives of Virology 149: 653-672. 2004, Parede, L., et al. Avian Pathology 32: 511-518. 2003, Rudd, M. F., et al. Archives of Virology 147: 1303-1322. 2002, Zierenberg, K., et al. Archives of Virology 145: 113-125. 2000, Zierenberg, K., et al. Avian Pathology 30: 55-62. 2001) and sequences obtained by sequencing the VP2 gene of seventeen vvIBDV strains submitted to our laboratory under import permit #44226. Regions of the VP2 gene were selected based on nucleotide mutations unique to the vvIBDV strains.

LightCycler technology uses probe pairs to identify nucleotide mutations (Bernard, P. S., et al. American Journal of Pathology 153: 1055-1061. 1998). Each pair consisted of a mutation probe, designed to detect point mutations, located over the site of the unique nucleotide region and an anchor probe located in a more conserved region of the genome adjacent to the mutation probe The probes were labeled with fluorescein (FITC), Red 640 or Red 705 such that the FITC on one probe was adjacent to a Red label on its pair. The FITC and Red dyes create a fluorescence resonance energy transfer (FRET) that is detected in the LightCycler instrument when both probes are bound to the RT-PCR products (Bernard, P. S., et al. Mutation detection by fluorescent hybridization probe melting curves. In: Rapid cycle real-time PCR methods and applications. S. Meuer, C. Wittwer, and K. -I. nakagawara, eds, Spinger-Verlag, Berlin heidelberg, Germany. 11-20. 2001). Each probe pair was designed so the anchor probe had a melting temperature (Tm) approximately 10° C. higher than the mutation probe (Table 2). This insures dissociation of the mutation probe before the anchor probe during the melting point analysis that followed the real-time RT-PCR assay.

Data analysis. During the RT-PCR assay fluorescence at 640 λ or 705 λ was detected and recorded at the end of each annealing step when both mutation and anchor probes were bound to the RT-PCR products. This allowed amplification of the IBDV genome to be detected in real-time.

Following 40 cycles of PCR amplification, the reactions were cooled slowly to 35° C. and then warmed slowly to 90° C. During this period, dissociation of the mutation probe from the RT-PCR products caused a loss of fluorescence which was detected and used to calculate a Tm. The Tm for an exact sequence match for each mutation probe is listed in Table 2.

TABLE 2
Probe pairs tested in this study.

| | | |
|---|---|---|
| Mutation Probe | 5′-Red705-CTCAGCTAATATCG | Tm = 55° C. |
| vv232: | ATGC-3′, SEQ ID NO:4 | |
| Anchor Probe | 5′-AGGTGGGGTAACAATCACACT | Tm = 64° C. |
| vv232; | GT-FITC-3′, SEQ ID NO:5 | |
| Mutation Probe | 5′-CTTATACTGGGTGCTACCAT | Tm = 58° C. |
| vv256: | C-FITC-3, SEQ ID NO:8′ | |
| Anchor Probe | 5′-Red640-CCTTATAGGCTTTG | Tm = 67° C. |
| vv256: | ATGGGACTGCGG-3, | |
| | SEQ ID NO:9 | |

^{A}Melting temperatures (Tm) for each probe were determined using the TM Utility 1.5 from Idaho Technologies Inc.

The Tm means of the vvIBDV group and non-vvIBDV group were analyzed for each probe using a one-way ANOVA.

Nucleotide sequence analysis. To validate the real-time RT-PCR results, 18 viruses submitted to our laboratory as suspect vvIBDV isolates were chosen for sequence analysis. Viruses were amplified using our standard RT-PCR procedures (Jackwood, D. J., et al. Avian Diseases 45: 330-339. 2001) and these RT-PCR products were purified using a Geneclean Spin Kit (BIO 101, Vista, Calif.) according to the manufacturer's instructions. The purified RT-PCR products were then sent to the University of Wisconsin Biotechnology Center DNA Sequence Facility (Madison, Wis.) for nucleotide sequencing. The nucleotide sequences were downloaded using Chromas (Technelysium Pty Ltd., Queensland, Australia) and analyzed using Omega software (Oxford molecular, Campbell, Calif.). The GenBank accession numbers of these sequences are listed as a set starting with AY906997 and ending with AY907014.

### Results

vvIBDV genetic markers. To design probe pairs for the real-time RT-PCR assay an analysis of published vvIBDV sequences was conducted to determine potentially unique nucleotide mutations. A rather large list of very virulent viruses was compared from numerous countries and continents. Based on these sequences three regions were identified with consistent mutations. Mutation and anchor probes were designed to these regions. Mutation probe vv232 was designed to exploit three silent mutations at nucleotide positions 827, 830 and 833. The second probe, vv256, covered nucleotides 894 to 914 and was designed to detect a nucleotide mutation that results in Valine at position 256 in non-vvIBDV and Isoleucine in vvIBDV. Two silent mutations at nucleotide positions 905 and 908 were also included in this probe.

Real-time RT-PCR. Both vvIBDV and non-vvIBDV strains were amplified in the real-time RT-PCR assay. The vv232 and vv256 probes hybridized to all viruses during this assay and produced a FRET signal during the annealing step (data not shown).

A Tm was calculated for the vv232 and vv256 probes with each vvIBDV sample. Initially we tested 18 IBDV samples that had been submitted to our laboratory as suspect vvIBDV strains (Table 3). The Tm values were reported as the mean of at least 2 but usually 3 or 4 separate real-time RT-PCR assays. The melting temperatures calculated using the vv232 probe were within two standard deviations of the Tm calculated for an exact sequence match with 17 of the 18 suspect vvIBDV samples. The Thai 4 sample had a 46.11° C. Tm which was considerably lower than expected for a vvIBDV strain. The vv256 probe results were similar except for the Thai 4 virus again (Tm=46.15° C.) and two additional viruses SA2 and 182 where the Tm values were slightly lower than expected 49.99 and 48.81° C., respectively.

TABLE 3
Mean Tm values for vv232 and vv256 probes
on samples suspected of being vvIBDV.

| Suspect | vv232^{A} | vv256^{B} |
|---|---|---|
| vvIBDV Strains | Mean Tm ± SD | Mean Tm ± SD |
| 183 | 53.94 ± 0.39 | 56.51 ± 0.47 |
| 9596 | 55.67 ± 0.17 | 56.08 ± 0.51 |
| AK2 | 54.81 ± 0.68 | 55.66 ± 0.85 |
| AL 10 | 54.49 ± 0.06 | 55.90 ± 0.44 |
| AL 13 | 54.41 ± 0.06 | 56.12 ± 0.48 |
| AL 4 | 53.88 ± 0.30 | 56.27 ± 0.48 |
| DR 4 | 55.73 ± 0.43 | 56.50 ± 0.51 |
| Isr 30 | 54.16 ± 0.09 | 54.31 ± 0.05 |
| Isr 4 | 54.01 ± 0.28 | 58.67 ± 1.71 |
| Isr 7 | 53.97 ± 0.11 | 58.00 ± 1.32 |
| Spain 1 | 53.77 ± 0.02 | 57.66 ± 1.49 |
| Jordan E | 56.24 ± 1.14 | 55.23 ± 0.41 |
| FD7 | 55.05 ± 0.54 | 55.04 ± 0.39 |
| 179 | 56.34 ± 0.01 | 55.00 ± 0.29 |
| SA 2 | 51.98 ± 1.50 | 49.99 ± 0.71 |
| Isr 13 | 54.29 ± 0.67 | 54.49 ± 0.55 |
| 182 | 54.46 ± 0.31 | 48.81 ± 0.53 |
| Thai 4 | 46.11 ± 0.06 | 46.15 ± 0.21 |

^{A}The mean melting temperature (Tm) and standard deviation (SD) obtained with probe vv232.
^{B}The mean melting temperature (Tm) and standard deviation (SD) obtained with probe vv256.

Assay validation. To further validate the vv232 and vv256 probes, 26 additional samples submitted to our laboratory as suspect vvIBDV and 18 known non-vvIBDV strains were examined (Table 4). The melting temperatures for each of the suspect vvIBDV were always above 52° C. and in all cases within one or two degrees of the Tm expected for an exact sequence match with the vv232 or vv256 probes. All non-vvIBDV strains tested had Tm values below 49° C.

TABLE 4
Validation of vv232 and vv256 probes.

| | vv232 Probe^{B} | vv256 Probe^{C} |
|---|---|---|
| | Mean Tm ± SD | Mean Tm ± SD |
| Suspect | | |
| vvIBDV Strains^{A} | | |
| 9664 | 54.89 ± 1.17 | 55.95 ± 0.67 |
| 91108 | 54.11 ± 0.79 | 56.13 ± 0.44 |
| AL 1 | 54.22 ± 0.64 | 56.25 ± 0.50 |
| AL 6 | 53.33 ± 0.37 | 56.22 ± 0.48 |
| Isr 1 | 54.06 ± 0.49 | 56.54 ± 2.29 |
| Isr 2 | 55.20 ± 0.56 | 56.58 ± 0.85 |
| Isr 3 | 54.94 ± 1.01 | 57.22 ± 1.17 |
| Isr 5 | 55.08 ± 0.61 | 56.37 ± 0.71 |
| Isr 6 | 55.28 ± 0.77 | 56.93 ± 1.02 |
| Isr 8 | 54.56 ± 1.28 | 57.09 ± 1.36 |
| Isr 9 | 54.55 ± 1.48 | 56.95 ± 1.20 |
| Isr 10 | 54.45 ± 0.78 | 56.58 ± 1.36 |
| Isr 11 | 54.31 ± 1.49 | 56.99 ± 1.22 |
| Isr 12 | 54.06 ± 1.16 | 56.41 ± 2.04 |
| Isr 14 | 54.93 ± 0.94 | 56.64 ± 1.53 |
| Isr 15 | 54.55 ± 1.01 | 56.83 ± 1.24 |
| Isr 16 | 55.16 ± 1.05 | 56.30 ± 0.71 |
| Isr 17 | 55.24 ± 0.71 | 56.46 ± 0.71 |
| Isr 19 | 54.89 ± 0.45 | 56.10 ± 0.46 |
| Isr 20 | 55.04 ± 1.01 | 56.33 ± 0.08 |
| Isr 21 | 54.93 ± 0.39 | 56.15 ± 0.39 |
| Isr 23 | 55.20 ± 0.11 | 56.32 ± 0.10 |
| Isr 24 | 53.92 ± 0.93 | 55.74 ± 0.08 |
| Isr 25 | 54.23 ± 1.15 | 56.18 ± 0.20 |
| Isr 28 | 52.86 ± 0.66 | 54.88 ± 0.78 |
| Isr 29 | 54.14 ± 0.72 | 55.19 ± 0.74 |
| Non-vvIBDV | | |
| Strains^{D} | | |
| Del E | 45.80 ± 0.16 | 45.65 ± 0.19 |
| D78 | 33.93 ± 0.72 | 46.67 ± 0.56 |
| STC | 45.29 ± 0.33 | 44.97 ± 0.28 |
| F16 | 45.33 ± 0.28 | 45.19 ± 0.23 |
| FDG | 45.56 ± 0.06 | 45.34 ± 0.24 |
| FDH | 46.30 ± 0.44 | 46.26 ± 0.59 |
| GA234 | 45.80 ± 0.39 | 46.10 ± 0.38 |
| MO196 | 46.65 ± 0.06 | 46.81 ± 0.21 |
| MS203 | 45.33 ± 0.39 | 45.88 ± 0.70 |
| T1 | 48.49 ± 0.33 | 48.30 ± 0.24 |
| AL186 | 45.84 ± 0.33 | 46.07 ± 0.26 |
| AR113 | 46.66 ± 0.28 | 47.03 ± 0.53 |
| WI240 | 47.32 ± 0.34 | 47.28 ± 0.34 |
| AR272 | 45.84 ± 0.33 | 45.96 ± 0.18 |
| AR 80 | 45.80 ± 0.05 | 46.27 ± 0.49 |
| AR84 | 45.92 ± 0.54 | 46.36 ± 0.14 |
| F15 | 41.97 ± 0.06 | 42.02 ± 0.12 |
| GA129 | 36.91 ± 1.93 | 39.39 ± 0.72 |

^{A}Validation of the vv232 and vv256 probes was conducted using 26 suspected vvIBDV strains.
^{B}The mean melting temperature (Tm) and standard deviation (SD) obtained with probe vv232.
^{C}The mean melting temperature (Tm) and standard deviation (SD) obtained with probe vv256.
^{D}Validation of the vv232 and vv256 probes was conducted using 18 known non-vvIBDV strains from the U.S.

The overall mean and standard deviation for all vvIBDV samples tested using the vv232 probe was 54.54±0.80° C. In contrast, the overall mean and standard deviation for the non-vvIBDV strains including Thai 4, using this probe was 44.78±3.55° C. These values were significantly different using ANOVA (p<0.01). Similarly, the mean and standard deviation for all vvIBDV and non-vvIBDV strains using the vv256 probe was 55.94±1.69 and 45.67±1.96° C., respectively. When compared using ANOVA the vv256 Tm values for vvIBDV and non-vvIBDV groups were also significantly different (p<0.01).

Since the vv232 probe pair was labeled with Red 705 and the vv256 probe pair was labeled with Red 640, they could be combined in one LightCycler reaction. The results obtained when the probes were combined were essentially identical to the results obtained when they were used separately (data not shown).

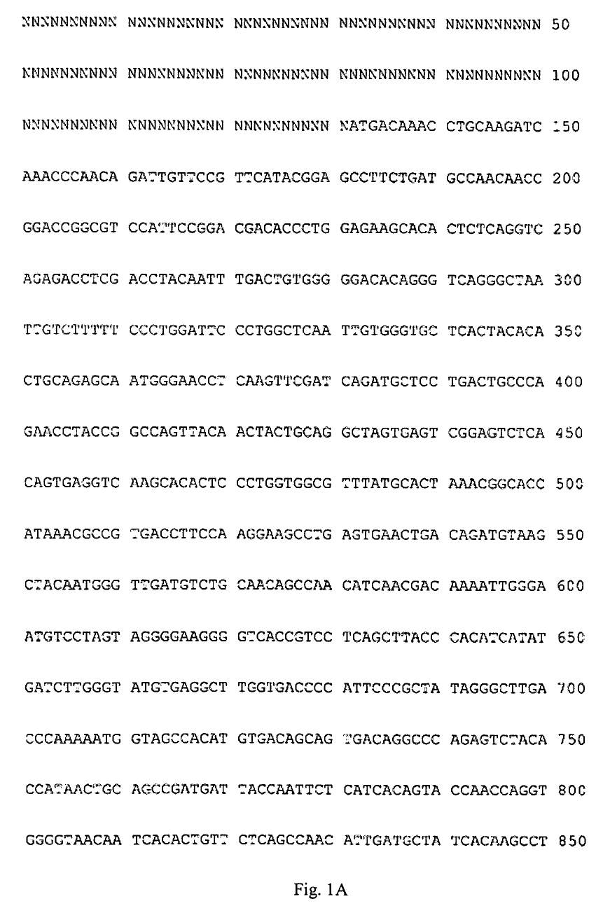

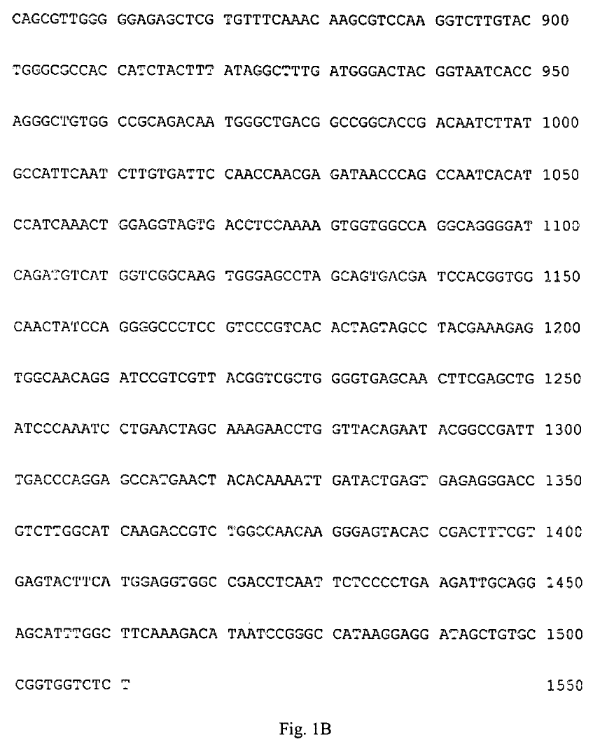

Nucleotide sequence analysis. The nucleotide sequence results for the 17 vvIBDV samples and 19 non-vvIBDV viruses correlated with the Tm values observed. FIGS. 1 and 2 list the nucleotide sequences of the mutation probes, the corresponding sequences of the 17 vvIBDV samples, 18 known non-vvIBDV strains and the Thai 4 sample. Sequence mutations were observed between the mutation probes and some vvIBDV strains. These mutations lowered the Tm values for these particular viruses but in only two samples (182 and SA2) using the vv256 probe were the Tm values below 50° C. In contrast, Tm values for Thai 4 and the 18 non-vvIBDV strains were always below 49° C. regardless of the probe used.

### Discussion

A real-time RT-PCR assay was developed and Tm analysis following this assay distinguished vvIBDV from non-vvIBDV strains. Samples were submitted to our laboratory as suspect vvIBDV strains because the flock history included high morbidity and mortality. Since only genetic material could be imported from outside the U.S. (import permit #44226) we were unable to confirm the vvIBDV phenotype using challenge studies. Thus, a genetic assay was developed that identified specific nucleotide sequences unique to vvIBDV strains. Although the exact genetic elements needed for expression of the very virulent phenotype have not been determined, our assay exploited two regions of the VP2 gene that contained 6 nucleotide mutations unique to these viruses. Probe pairs vv232 and vv256 successfully hybridized to the vvIBDV RT-PCR products and produced a FRET signal in the LightCycler. When the vv232 and vv256 probes were combined, we were able to obtain Tm data for both probe pairs in a single reaction; reducing costs of the assay and the length of time needed to obtain results.

Melting temperature analysis indicated that probes vv232 and vv256 could distinguish vvIBDV strains from non-vvIBDV strains. Using the vv232 probe, the mean Tm for all the vvIBDV samples tested was 54.54° C. which was within a half degree of the predicted Tm for an exact vvIBDV sequence match. Although submitted as a suspect vvIBDV, our results with both vv232 and vv256 probes indicated that the Thai 4 sample was not a very virulent strain.

Nucleotide sequencing of 17 vvIBDV strains confirmed the Tm results and their sequences were nearly identical to previously identified vvIBDV strains. Only the Jordan E virus had a point mutation in the region of the vv232 probe. This mutation did not markedly lower the Tm for this virus and probe but a large standard deviation (±1.41° C.) was observed suggesting more than one virus may have been present in the sample. Our previous studies indicated that genetic quasispecies are frequently found in field isolates of IBDV (Jackwood, D. J. and S. E. Sommer. Vir 304: 105-113. 2002).

Point mutations were observed in 7 of the 17 viruses sequenced across the vv256 probe region. Each of the 7 viruses had only one point mutation which did not noticeably lower their Tm with this probe except in two cases (SA2 and 182). It is not clear why a single mutation in these two viruses lowered their Tm with probe vv256 when this was not the case with the other 5 viruses that contained single mutations. If genetic quasispecies were present in this sample and the nucleotide sequence of the dominate viral population was determined, it is possible that subordinate quasispecies populations in these 5 viruses contributed to a higher Tm than was expected by a relatively pure culture of viruses with a single mutation across the vv256 probe region.

Our results demonstrates that a Tm value for one or both probes above 51° C. can be used to identify vvIBDV. Only two vvIBDVs had Tm values below 51° C. using the vv256 probe and none had values below this using the vv232 probe. Using this cut-off value and both probes in the real-time RT-PCR assay, helps insure that viruses like SA2 and 182 would have been accurately identified as vvIBDV strains since their Tm values using the vv232 probe were 51.98 and 54.46° C., respectively. Furthermore, all the non-vvIBDV strains tested had Tm values below 49° C. with both probes. Tm differences observed using the vv232 and vv256 probes were statistically significant between vvIBDV and non-vvIBDV strains at p<0.01.

Each mutation probe was designed to detect 3 nucleotides unique to vvIBDV strains; a total of 6 unique nucleotides. An amino acid at position 256 (Ile) is unique to all vvIBDV strains (Liu, H. J., et al. Research in Veterinary Science 70: 139-147. 2001, Parede, L., et al. Avian Pathology 32: 511-518. 2003). One nucleotide in our vv256 probe exploits this unique vvIBDV sequence. The 5 other unique nucleotides detected by our probes, do not affect the amino acid sequence of VP2 but they are evolutionarily unique to vvIBDV strains. Targeting 6 nucleotide mutations with both probes reduces the probability of misdiagnosis due to random mutation. This was demonstrated with the Jordon E virus which had single mutations in the regions targeted by both probes.

Results obtained with a mutation probe designed to hybridize with a third mutated sequence encompassing nucleotides 784 to 801 of the VP2 gene of the vvIBDV strains and an anchor probe directed at a sequence downstream of the third mutated sequence did not identify a nucleotide sequence responsible for the Alanine substitution mutation at amino acid 222 in vvIBDV strains. Although this Alanine mutation is unique to all vvIBDV strains sequenced to date (Banda, A. and P. Villegas. Avian Diseases 48: 540-549. 2004, Brown, M. D. and M. A. Skinner. Virus Research 40: 1-15. 1996, Chen, H. Y., et al. Avian Diseases 42: 762-769. 1998, Domanska, K., et al. Archives of Virology 149: 465-480. 2004, Indervesh, A. K., et al. Acta virologica 47: 173-177. 2003, Kwon, H. M., et al. Avian Diseases 44: 691-696. 2000, Lin, Z., et al. Avian Diseases 37: 315-323. 1993, Liu, H. J., et al. Research in Veterinary Science 70: 139-147. 2001, Owoade, A. A., et al. Archives of Virology 149: 653-672. 2004, Parede, L., et al. Avian Pathology 32: 511-518. 2003, Rudd, M. F., et al. Archives of Virology 147: 1303-1322. 2002, Zierenberg, K., et al. Archives of Virology 145: 113-125. 2000, Zierenberg, K., et al. Avian Pathology 30: 55-62. 2001) the mutation and anchor probes to this third mutated sequence did not produce accurate or reliable data.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of identifying an animal infected with very virulent infectious bursal virus (vvIBDV),
contacting a nucleic acid test sample with a first mutation probe designed to hybridize with a first mutated target sequence encompassing nucleotide 827 through nucleotide 833 of the sense strand of the VP2 gene of vvIBDV, or the reverse complement thereof, and with a first anchor probe designed to hybridize with a first anchor sequence that is from 0 to 5 nucleotides upstream of the first mutated target sequence;
wherein the nucleic acid test sample is double-stranded RNA obtained from the animal, a cDNA derived from the double-stranded RNA, a single strand of the double stranded RNA, or a single strand of the cDNA, or any combination thereof;
wherein the first mutation probe is 12 or more nucleotides in length and comprises the sequence TAATATC, SEQ ID NO: 2, or the reverse complement thereof;
wherein the first anchor probe is 12 or more nucleotides in length and disassociates from its complementary sequence at a temperature at least 4° C. higher than the temperature at which the first mutation probe disassociates from its complementary sequence; and
wherein the first mutation probe and the first anchor probe are contacted with the nucleic acid test sample under conditions that allow the first mutation probe and first anchor probe to hybridize with one or the other strand of the VP2 gene of a vvIBDV to provide a first hybridization complex; and
determining the temperature at which the first mutation probe disassociates from the first hybridization complex;
wherein one of the first mutation probe and the first anchor probe is labeled with an acceptor fluorophore and the other of the first mutation probe and the first anchor probe is labeled with a donor fluorophore of a fluorescence energy transfer pair;
wherein formation of the first hybridization complex and disassociation of the first hybridization complex is determined by fluorescence resonance energy transfer (FRET) analysis, and
wherein disassociation of the first mutation probe from the first hybridization complex at a temperature greater than the temperature at which the first mutation probe disassociates from SEQ ID NO:1, or at a temperature within 4° C. of the temperature at which the first mutation probe disassociates from its complementary sequence, or both indicates that the animal is infected with vvIBDV.

2. The method of claim 1, further comprising the step of comparing the temperature at which the first hybridization complex melts with the melting temperature of a hybridization complex formed when the first mutation probe is hybridized with a nucleic acid comprising the targeted portion of SEQ ID NO: 1, or its reverse complement.

3. The method of claim 1, further comprising the step of comparing the temperature at which the first hybridization complex melts with the melting temperature of a hybridization complex formed when the first mutation probe is hybridized with a nucleic acid comprising its complementary sequence.

4. The method of claim 1, wherein the first mutation probe comprises SEQ ID NO: 4, and wherein a melting temperature of 49° C. or greater for a hybridization complex comprising said mutation probe and a nucleic acid in the test sample indicates that the animal is infected with vvIBDV.

5. The method of claim 1, wherein the first mutation probe comprises from 12 to 18 consecutive nucleotides in SEQ ID NO: 4, or the reverse complement thereof.

6. The method of claim 1, wherein the nucleic acid sample comprises a cDNA product obtained by reverse transcriptase-polymerase chain reaction (RT-PCR) amplification of a double-stranded RNA sample obtained from the animal.

7. The method of claim 1, wherein the RNA sample is obtained from an avian species.

8. The method of claim 1, wherein the RNA sample is obtained from a chicken.

9. The method of claim 1, wherein the RNA sample is obtained from the bursa of a chicken.

10. The method of claim 1, wherein the melting temperature of a hybridization complex comprising the first mutation probe and its complementary sequence is at least 10° C. lower than the melting temperature of a hybridization complex comprising the first anchor probe and its complementary sequence.

11. The method of claim 1, further comprising contacting the nucleic acid test sample with a second mutation probe designed to hybridize with a second mutated target sequence encompassing nucleotide 897 through nucleotide 908 of the VP2 gene of vvIBDV, or the reverse complement thereof, and with a second anchor probe designed to hybridize with a second anchor target sequence that is within 0 to 5 nucleotides downstream of the second mutated target sequence;
wherein the second mutation probe is 12 or more nucleotides in length and comprises the sequence, ATACTGGGTGCT, SEQ ID NO: 6, or the reverse complement thereof;
wherein the second anchor probe is at least 12 nucleotides in length and disassociates from its complementary sequence at a temperature at least 4° C. higher than the temperature at which the second mutation probe disassociates from its complementary sequence;
wherein the second mutation probe and the second anchor probe are contacted with the nucleic acid test sample under conditions that allow the second mutation probe and the second anchor probe to hybridize with a strand of the VP2 gene of vvIBDV to provide a second hybridization complex; and
determining the temperature at which the second mutation probe disassociates from the second hybridization complex;
wherein one of the second mutation probe and the second anchor probe is labeled with an acceptor fluorophore and the other of the second mutation probe and the second anchor probe is labeled with a donor fluorophore of a fluorescence energy transfer pair;
wherein formation of hybridization complex and disassociation of the second hybridization complex is determined by FRET analysis; and
wherein disassociation of the second mutation probe from the second hybridization complex at a temperature greater than the temperature at which the second mutation probe disassociates from SEQ ID NO: 1, or at a temperature within 4° C. of the temperature at which the second mutation probe disassociates from its complementary sequence, or both indicates that the animal is infected with vvIBDV.

12. The method of claim 11, wherein the nucleic acid sample comprises a cDNA product obtained by reverse-transcriptase polymerase chain reaction (RT-PCR) amplification of a double-stranded RNA sample obtained from the animal.

13. The method of claim 11, wherein the RNA sample is obtained from the bursa of a chicken.

14. The method of claim 11, wherein the melting temperature of a hybridization complex comprising the second mutation probe and its complementary sequence is at least 10° C. lower than the melting temperature of a hybridization complex comprising the second anchor probe and its complementary sequence.

15. A method of identifying an animal infected with very virulent infectious bursal virus (vvIBDV),
contacting a nucleic acid test sample with a mutation probe designed to hybridize with a mutated target sequence encompassing nucleotide 897 through nucleotide 908 of the VP2 gene of vvIBDV, or the reverse complement thereof, and with an anchor probe designed to hybridize with an anchor target sequence that is within 0 to 5 nucleotides downstream of the mutated target sequence;
wherein the nucleic acid test sample is double-stranded RNA obtained from the animal, a cDNA derived from the double-stranded RNA, a single strand of the double stranded RNA or a single strand of the cDNA, or any combination thereof;
wherein the mutation probe is 12 or more nucleotides in length and comprises the sequence, ATACTGGGTGCT, SEQ ID NO: 6, or the reverse complement thereof;
wherein the anchor probe is at least 12 nucleotides in length and disassociates from its complementary sequence at a temperature at least 4° C. higher than the temperature at which the mutation probe disassociates from its complementary sequence;
wherein the mutation probe and the anchor probe are contacted with the nucleic acid test sample under conditions that allow the probes to hybridize with at least one strand of a nucleic acid in the test sample to provide a hybridization complex; and
determining the temperature at which the mutation probe disassociates from the hybridization complex;
wherein one of the mutation probe and the anchor probe is labeled with an acceptor fluorophore and the other of the mutation probe and the anchor probe is labeled with a donor fluorophore of a fluorescence energy transfer pair;
wherein formation of hybridization complex and disassociation of the hybridization complex is determined by FRET analysis, and
wherein disassociation of the mutation probe from the hybridization complex at a temperature greater than the temperature at which the mutation probe disassociates from a nucleic acid comprising the targeted sequence in SEQ ID NO:1 or the reverse complement thereof, or at a temperature within 4° C. of the temperature at which the mutation probe disassociates from its complementary sequence, or both indicates that the animal is infected with vvIBDV.

16. The method of claim 15, further comprising the step of comparing the temperature at which the hybridization complex melts with the melting temperature of a hybridization complex formed when the mutation probe is hybridized with a nucleic acid comprising the targeted portion of SEQ ID NO: 1, or its reverse complement.

17. The method of claim 15, further comprising the step of comparing the temperature at which the hybridization complex melts with the melting temperature of a hybridization complex formed when the mutation probe is hybridized with a nucleic acid comprising its complementary sequence.

18. The method of claim 15, wherein the mutation probe comprises the sequence of SEQ ID NO: 8, and wherein a disassociation temperature of 51° C. or greater for the mutation probe from a nucleic acid in the sample indicates that the animal is infected with vvIBDV.

19. The method of claim 15, wherein the mutation probe comprises from 12 to 23 contiguous nucleotides in SEQ ID NO: 8, or the reverse complement thereof.

20. The method of claim 15, wherein the double stranded sample is a cDNA product obtained by reverse transcriptase-polymerase chain reaction (RT-PCR) amplification of a double-stranded RNA sample obtained from the animal.

21. The method of claim 15, wherein the RNA sample is obtained from the bursa of a chicken.

22. The method of claim 15, wherein the melting temperature of a hybridization complex comprising the mutation probe and its complementary sequence is at least 10° C. lower than the melting temperature of a hybridization complex comprising the anchor probe and its complementary sequence.

23. A kit for identifying animals infected with vvIBDV, comprising:
at least one of:
a) a first pair of oligonucleotide probes,
wherein one of said first pair of oligonucleotide probes comprises a mutation probe that is complementary to a first mutated target sequence encompassing nucleotide 827 through nucleotide 833 of the VP2 gene of vvIBDV, or the reverse complement thereof;
wherein the mutation probe is 12 or more nucleotides in length and comprises the sequence TAATATC, SEQ ID NO: 2, or the reverse complement thereof;
wherein the other of said first pair of oligonucleotide probes comprises an anchor probe that is identical to a first anchor target sequence that is from 0 to 5 nucleotides upstream of the first mutated target sequence, or the reverse complement thereof;
wherein the first anchor probe is 12 or more nucleotides in length and disassociates from its complementary sequence at a temperature at least 4° C. greater than the temperature at which the first mutation probe disassociates from its complementary sequence; and
b) a second pair of oligonucleotide probes,
wherein one of said second pair of oligonucleotide probes is a second mutation probe comprising a sequence complementary to a second mutation target sequence encompassing nucleotide 897 through nucleotide 908 of the VP2 gene of vvIBDV, or the reverse complement thereof;
wherein the second mutation probe is 12 or more nucleotides in length and comprises the sequence, ATACTGGGTGCT, SEQ ID NO: 6, or the reverse complement thereof; and
wherein the other of said second pair of oligonucleotide probes is an anchor probe that is identical to a second anchor target sequence that is from 0 to 5 nucleotides downstream of the first mutated target sequence, or the reverse complement thereof;
wherein the second anchor probe is 12 or more nucleotides in length and disassociates from its complementary sequence at a temperature at least 4° C. greater than the temperature at which the second mutation probe disassociates from its complementary sequence.

24. The kit of claim 23, wherein for each pair of oligonucleotide probes one member of the pair is labeled with an acceptor fluorophore of a fluorescence energy transfer pair and the other member is labeled with a donor fluorophore of a fluorescence energy transfer pair.

25. The kit of claim 23, wherein said kit comprises the first pair of oligonucleotide probes and the second pair of oligonucleotide probes.

26. The kit of claim 24, wherein the emission of the acceptor fluorophore on the labeled oligonucleotide of one pair is different from the emission of the acceptor fluorophore on the labeled oligonucleotide of the other pair.

27. A method for detecting the VP2 gene of a vvIBDV or a portion thereof in a nucleic acid sample comprising:
contacting the nucleic acid sample with one or both of the oligonucleotide pairs of claim 20;
determining the temperature at which a hybridization complex formed between the first mutation probe and a nucleic acid in the sample, or a hybridization complex formed between the second mutation probe and a nucleic acid in the sample, or both hybridization complexes melt, and comparing the melting temperature of each of the hybridization complexes to one or both of the following:
a first control hybridization complex comprising all or a portion of the VP2 gene of a non-very virulent IBDV and one or both of the mutation probes, and
a second control hybridization complex comprising all or a portion of the VP2 gene of a vvIBDV and one or both of the mutation probes;
wherein samples that produce hybridization complexes whose melting temperatures are greater than the first and/or the second control hybridization complex contain the VP2 gene or a portion thereof of a vvIBDV.

28. The method of claim 27, wherein the determination is made during real-time RT-PCR.
